SEQUENCE LISTING (14 sequences)

SEQ ID NO: 1 - DNA, 2350 bp, homo sapiens
cattgctcttcagcaccagggttctggacagcgccccaagcaggcagctgatcgcacgccccttcctctcaatctccgccagcgctgctactgcccctctagtaccccctgctgcagagaaagaatattacaccgggatccatgcagccagcaatgatgatgttttccagtaaatactgggcacggagagggttttccctggattcagcagtgcccgaagagcatcagctacttggcagctcaacactaaataaacctaactctggcaaaaatgacgacaaaggcaacaagggaagcagcaaacgtgaagctgctaccgaaagtggcaagacagcagttgttttctccttgaagaatgaagttggtggattggtaaaagcactgaggctctttcaggaaaaacgtgtcaacatggttcatattgaatccaggaaatctcggcgaagaagttctgaggttgaaatctttgtggactgtgagtgtgggaaaacagaattcaatgagctcattcagttgctgaaatttcaaaccactattgtgacgctgaatcctccagagaacatttggacagaggaagaagagctagaggatgtgccctggttccctcggaagatctctgagttagacaaatgctctcacagagttctcatgtatggttctgagcttgatgctgaccacccaggatttaaggacaatgtctatcgacagagaagaaagtattttgtggatgtggccatgggttataaatatggtcagcccattcccagggtggagtatactgaagaagaaactaaaacttggggtgttgtattccgggagctctccaaactctatcccactcatgcttgccgagagtatttgaaaaacttccctctgctgactaaatactgtggctacagagaggacaatgtgcctcaactcgaagatgtctccatgtttctgaaagaaaggtctggcttcacggtgaggccggtggctggatacctgagcccacgagactttctggcaggactggcctacagagtgttccactgtacccagtacatccggcatggctcagatcccctctacaccccagaaccagacacatgccatgaactcttgggacatgttccactacttgcggatcctaagtttgctcagttttcacaagaaataggtctggcgtctctgggagcatcagatgaagatgttcagaaactagccacgtgctatttcttcacaatcgagtttggcctttgcaagcaagaagggcaactgcgggcatatggagcaggactcctttcctccattggagaattaaagcacgccctttctgacaaggcatgtgtgaaagcctttgacccaaagacaacttgcttacaggaatgccttatcaccaccttccaggaagcctactttgtttcagaaagttttgaagaagccaaagaaaagatgagggactttgcaaagtcaattacccgtcccttctcagtatacttcaatccctacacacagagtattgaaattctgaaagacaccagaagtattgaaaatgtggtgcaggaccttcgcagcgacttgaatacagtgtgtgatgctttaaacaaaatgaaccaatatctggggatttgatgcctggaactatgttgttgccagcatgatctttttggggcttagcagcagttcagtcaatgtcatataacgcaaataaccttctgtgtcatggcttggctaataagcatgcaattccatatatctataccatcttgtaactcactgtgttagtatataaagcaccataagaaatccaatggcagataacctgaaataacgtattatgtttaaacatcttaaaaagatttgacattcctgcttagtgtccttaaccaaactgcatctagttaaaatttgtaacaaatagccctcttatgagtctcatttatgcccttttctttttcagatctaagcctttcctctgtgttcattagataaaatgaaaaaaagcagtgaagctgtttccattttcaatagtatcagtgttttcacgcattatttgagataaacccagaattgtaggaaacttcccatcacaataacaaaggttcaatattctatttcaaaaattgttgaggtaacacagcagttggaatgatttttaggttgagtatttacacaatgcaagaaaacacctttttacaaatggaattatgtaggttgcgttgaccttgtagaacctgagttatgacaagcttcctgaagtattttggaagatagtacttccggaaaggacattaggaaagactaaacagtggacaatcaatcttgggactatgaattttatgctggaataaagtaaattatcatgttc

SEQ ID NO: 2 - protein, 490 aa, homo sapiens
MQPAMMMFSSKYWARRGFSLDSAVPEEHQLLGSSTLNKPNSGKNDDKGNKGSSKREAATESGKTAVVFSLKNEVGGLVKALRLFQEKRVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGI

SEQ ID NO: 3 - DNA, 2638 bp, mus musculus
cactgctcttcagcaccagggttctggacagcgccccgagcaggcagctgccactgcagttcctccttcatctctgccaaggccgcccctctggtcccccctgctgctgagaaagaaaattacatcgggagccatgcagcccgcaatgatgatgttttccagtaaatactgggccaggagagggttgtccttggattctgctgtgccagaagatcatcagctacttggcagcttaacacaaaataaggctatcaaaagcgaggacaagaaaagcggcaaagagcccggcaaaggcgacaccacagagagcagcaagacagcagttgtgttctccttgaagaatgaagttggtgggctggtgaaagcacttagactattccaggaaaaacatgtcaacatgcttcatatcgaatccaggcggtcccggcgaagaagttctgaagtcgaaatcttcgtggactgcgaatgtggcaaaacggaattcaatgagctcatccagttgctgaaatttcagaccaccattgtgaccctgaatccgcctgagagcatttggacggaggaagaagatctcgaggatgtgccgtggttccctcggaagatctctgagttagacagatgctctcaccgagtcctcatgtacggcaccgagcttgatgccgaccatccaggatttaaggacaatgtctatcgacagaggaggaagtattttgtggatgtggccatgggctataaatatggtcagcccattcccagggtcgagtacacagaagaagagactaaaacttggggtgttgtgttccgggagctctccaaactctacccgactcatgcttgccgggagtacctgaaaaacctccccctgctgaccaagtactgtggctacagggaagacaacgtgccgcaactggaagacgtctccatgtttctgaaagagcgatctggcttcacagtgagaccagtggctggctacctgagcccaagagacttcctggcgggcctggcctacagagtattccactgcacccagtacgtgcggcatggctccgaccccctctacaccccggaaccagatacatgccatgaactcttgggacacgtgccactgcttgcggatcccaagtttgctcagttttcccaagagataggcttagcgtctctgggagcctcagatgaggacgttcagaaactagccacgtgctatttcttcacaatcgagttcggcctttgcaagcaagagggtcaactgcgggcgtatggagcagggttactttcgtccatcggagaattgaagcatgctctttccgacaaggcgtgtgtgaaatcctttgacccaaagacgacctgcttgcaggaatgcctaatcaccacctttcaggacgcttactttgtttcggacagttttgaagaagccaaagaaaagatgagggactttgcaaagtcaattacccgtcccttctcggtatacttcaaccgctacacgcagagcattgaaattctgaaagacaccagaagtattgagaatgtggtgcaggacctgcgcagtgatttgaacacagtgtgtgatgccttgaataaaatgaaccaatatctggggatttgatgcctagaaccagagttattgtcagcatgagctcttggggggtgtagcaacaatgcagtcaatgttatccaacatcaacaactttctgtgtcatggttggctagtaagcatgcaattctgtatgtccatacctctgtgtaacttaataacacaaaaatgctctaaagaacccatgcagataaccactcaccatttgaaagattgtgatcctatttggacatctcaagtagagttgacatttctgattagcgaacaaactgttaacttaagcaaactgtgactttgaaatctgtagcaaacattcctcgcacaattccagtcggtgagttgtggaacttttcttccttggacctgagactttcctctgtgttcattagataaaatgaaaatagttgggaggtggtttctattttcaatagtatccgtgttatttgagataaactagagttgctccacgctttgcatcacagcaacaaaggatttaatattctacttcagaagctgttcagaaacacagcagttgggatggatgtagactgagtgttcagacaatgcaagcaaagaaaagttttgataaacaggatatataggttgtactgacctcgttgaaaccaatttgtggcaagcttcctgaagagcttctggaaggaaacacttgaacaaagaatattcgggaagcttaaacagaagggatgaaaatcttggaactgtgaatgtattgttaggatagagtgaattatcactgcaggcttttgactccttttgcttagactgagaacctcaaatcccacagggatgtaaataccatctctgattccaaagagttggagacggagtcgtagagaaacaaagggatttgcttcagttaggtctgatgagatgtgccatggtcataagccactgcccttttatgttggacatctgacaagtctactgtagtgtacatgcatgtttatgtattgacacagaaagaaaattattgcttataaaatgaatgcttctcaataaacagaatcttgcccccaaaaaaaaaaaaaa

SEQ ID NO: 4 - protein, 488 aa, mus musculus
MQPAMMMFSSKYWARRGLSLDSAVPEDHQLLGSLTQNKAIKSEDKKSGKEPGKGDTTESSKTAVVFSLKNEVGGLVKALRLFQEKHVNMLHIESRRSRRRSSEVEIFVDCECGKTEFNELIQLLKFQTTIVTLNPPESIWTEEEDLEDVPWFPRKISELDRCSHRVLMYGTELDADHPGFKDNVYRQRRKYFVDVAMGYKYGQPIPRVEYTEEETKTWGVVFRELSKLYPTHACREYLKNLPLLTKYCGYREDNVPQLEDVSMFLKERSGFTVRPVAGYLSPRDFLAGLAYRVFHCTQYVRHGSDPLYTPEPDTCHELLGHVPLLADPKFAQFSQEIGLASLGASDEDVQKLATCYFFTIEFGLCKQEGQLRAYGAGLLSSIGELKHALSDKACVKSFDPKTTCLQECLITTFQDAYFVSDSFEEAKEKMRDFAKSITRPFSVYFNRYTQSIEILKDTRSIENVVQDLRSDLNTVCDALNKMNQYLGI

SEQ ID NO: 5 - DNA, 2581 bp, rattus norvegicus
cagggttctggacagcgcctcgagcagccagctgccgctcaccttcctcctacatctctgccaaggctgcccctctgatcccccctgctgctgagaaagaaaattacatcgggatccatgcagcccgcaatgatgatgttttccagtaaatactgggccaggagagggttgtccttggattcagcggtgccagaagagcatcagatacttggcggcttaacacaaaataaggctaccgctagcaaaagcgaggacaagagaagcggcaaagacacttcggagagcagcaagactgcggttgtgttctccctgaagaatgaagttggcgggctggtgagagcactgagactcttccaggaaaaacacgtcaacatgctccatattgaatccaggaggtcccggcgaagaagttctgaagtcgaaatcttcgtggactgtgaatgtggcaaaacagaattcaacgagctcattcagttgctcaagtttcagaccaccattgtgacgctgaatccacctgacaacatttggacggaggaagaagaactagaggatgtgccgtggttccctcggaagatctctgagttagacagatgctctcacagagtcctcatgtacggcaccgagcttgacgccgaccacccaggattcaaggacaacgtctatcgacagaggaggaagtattttgtggatgtggccatgggttataaatatggccagcccattcccagggtggaatacacagaagaagagactaaaacttggggtgttgtgtttcgggagctctccaaactctaccccactcatgcttgccgagagtacctgaaaaacttccccctgctgaccaagtactgcggctacagggaagacaacgtcccgcagctggaagacgtctccatgtttctgaaagagcgatctggcttcacagtgagaccagtggctggctacctgagcccaagagacttcctggctgggctggcctacagagtattccactgcactcagtacgtgcggcatggctccgaccccctctacaccccggaaccagacacatgccatgagctcctgggacatgtgccactgctggcggatcccaagttcgctcagttttctcaagaaataggcttagcctctctgggagcctcagatgaagacgttcagaaactggccacgtgctatttcttcacaatcgagttcggcctttgcaagcaagaaggtcaactgcgggcgtacggagcagggttactttcctccatcggagaattgaagcatgctctttctgacaaggcgtgtgtaaaagcctttgacccgaagacaacctgcttgcaggaatgcctaatcaccaccttccaagatgcttactttgtttctgaaagttttgaagaagccaaagagaagatgagggattttgcaaagtcaattacccgtcctttctcagtatatttcaacccctacacacagagcattgaaattctgaaagacaccagaagtattgagaatgtggtgcaggacctgcgcagtgatttgaacaccgtgtgcgacgccttgaataaaatgaaccaatatttggggatttgagcctattgtcagcacgagctcttgggggcttagcaacaatgcagtcaatgttatccaacatcaacaactttctgtgtcatggctggctagtaagcatgcaattccatgtgtctatacctctatgtaacttaacatacaaaaatgatctaagaaacccaggcagatgaccattcagcattttaaagattgtgatctatttgaacatctcaagtagatttgacatttctgattagtgagcaaactgtaacttaagcaaactgtgtctttaaaatttgtagccaacattcctcacacaattccagctgctgagtccttgaccttttcttccttggacctgagtctttcctctgtgttcattagataaaatgaaaacagttgggaggtggtttctactttcaatagtattggtgttctctgagataaactagagttgctccaagcttcgcatcacagtaacaaaagatttaatattttacttcagaagctgttcagaaacacagcgattggaatgaatctggactgagtgtttagacaatgcaagaaaagaaaaattttgataaacaggatatatagattgcactgaccttgttgaaaccaatttgtggtacgcttcctgaagtgcttttggaaggaaacactttgacaaagaatatttggaaaggttaaacagaagggaagaaaatcttggaactgtgaatgtgtcattagaataaagtgaattatcagtgcaggtgtgactcctttctcttacactgagaaccccaaatcctgcagggatgtgagtaccatctctgattccgaagatttggaaaccgagtcacagagaaacaaagggatttgcttcagttaggtctgttggctgggggtgcagtcataatccccccccccctttatgttggacttctggcaagtctactgtagtgtacatgcgggtttatgtatggacaaaaaaagaaaactaatgcgtataaaactaatgcttctcaataaacagaaacttgccccc

SEQ ID NO: 6 - protein, 485 aa, rattus norvegicus
MQPAMMMFSSKYWARRGLSLDSAVPEEHQILGGLTQNKATASKSEDKRSGKDTSESSKTAVVFSLKNEVGGLVRALRLFQEKHVNMLHIESRRSRRRSSEVEIFVDCECGKTEFNELIQLLKFQTTIVTLNPPDNIWTEEEELEDVPWFPRKISELDRCSHRVLMYGTELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYVRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQDAYFVSESFEEAKEKMRDFAKSITRPFSVYFNPYTQSIEILKDTRSIENVVQDLRSDLNTVCDALNKMNQYLGI

SEQ ID NO: 7 - DNA, 22 bp, mus musculus
gacatcggatcagaagactccc

SEQ ID NO: 8 - DNA, 21 bp, mus musculus
ctccctctttcggaggaatgg

SEQ ID NO: 9 - DNA, 23 bp, mus musculus
caccatgattgaagacaacaagg

SEQ ID NO: 10 - DNA, 23 bp, mus musculus
cgtgaattcaatcttgggaatgg

SEQ ID NO: 11 - DNA, 30 bp, mus musculus
ggttccctcggaagatctctgagttagaca

SEQ ID NO: 12 - DNA, 28 bp, mus musculus
agagctcccggaatacaacaccccaagt

SEQ ID NO: 13 - DNA, 26 bp, mus musculus
tgctcttcagcaccagggttctggac

SEQ ID NO: 14 - DNA, 25 bp, mus musculus
agaattgcatgcttactagccaacc

(19) United States
(12) Patent Application Publication
(10) Pub. No.: US 2006/0275759 A1
(43) Pub. Date: Dec. 7, 2006

(54) Neuronally expressed tryptophane hydroxylase and its use

(76) Inventors: Diego Walther, Berlin (DE); Michael Bader, Berlin (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/519,826

(22) PCT Filed: Jul. 16, 2003
(86) PCT No.: PCT/EP03/07744

§ 371 Date: Sep. 14, 2005

(30) Foreign Application Priority Data
Jul. 16, 2002 (DE) 102 32 151.5

(51) Int. Cl. C12Q 1/68; C07H 21/04; C12N 9/06; A61K 48/00; C12N 1/21; C07K 16/40

(52) U.S. Cl. 435/6; 536/23.2; 514/44; 530/388.26; 435/69.1; 435/191; 435/320.1; 435/325; 435/252.3

(57) ABSTRACT

The present invention relates to novel, specifically neuronal expressed proteins with tryptophane hydroxylase activity, nucleic acid sequences, recombinant nucleic acid molecules containing these nucleic acid sequences or vectors containing these nucleic acid sequences or the recombinant nucleic acid molecules encoding for a neuronal tryptophane hydroxylase. The invention also relates to transgenic organisms containing these nucleic acid sequences, the recombinant nucleic acid molecules or the above cited vectors. The invention moreover refers to mono- or polyclonal antibodies directed against the isolated proteins. Furthermore, the invention relates to the use of these nucleic acid sequences and proteins for diagnosis, predisposition, therapy and monitoring of neuronal diseases. Possible fields of application among others are medicine and the pharmaceutical industry.

### SUMMARY OF THE INVENTION

The present invention relates to novel, specifically neuronal expressed proteins with tryptophane hydroxylase activity, nucleic acid sequences, recombinant nucleic acid molecules containing these nucleic acid sequences or vectors containing these nucleic acid sequences or the recombinant nucleic acid molecules encoding for a neuronal tryptophane hydroxylase.

The invention also relates to transgenic organisms containing these nucleic acid sequences, the recombinant nucleic acid molecules or the above cited vectors. The invention moreover relates to mono- or polyclonal antibodies directed against the isolated proteins.

Furthermore, the invention relates to the use of these nucleic acid sequences and proteins for diagnosis, predisposition, therapy and monitoring of neuronal diseases. Possible fields of application among others are medicine and the pharmaceutical industry.

### STATE OF THE ART

Serotonin not only is a neurotransmitter in the central nervous system (CNS), but also a hormone, which is found all over in the periphery and takes part in vasoconstriction and thrombocyte function. Tryptophane hydroxylase is the enzyme determining the velocity of the serotonin biosynthesis (velocity-controlling enzyme). Serotonin (5-hydroxytryptamine, 5-HT) is a monoaminergic neurotransmitter, which is—together with tryptophane hydroxylase (TPH; EC 1.14.16.4) as the velocity-controlling enzyme in the first stage of serotonin synthesis (P. F. Fitzpatrick, Annu. Rev. Biochem. 68, 355 (1999))—involved in several aspects of mood-control and sleep regulation, of anxiety, alcoholism, drug abuse, food uptake and sexual behaviour (J. Veenstra-VanderWeele, G. M. Anderson, E. H. Cook Jr, Bur. J. Pharmacol. 410, 165 (2000)). The TPH-enzyme, together with phenylalanine (PAH)- and tyrosine hydroxylases (TH), belongs to the superfamily of aromatic amino acid hydroxylases (P. F. Fitzpatrick, Annu. Rev. Biochem. 68, 355 (1999)).

The serotonergic projection system is the most extensive monoaminergic system in the vertebrate brain, however it is also the system the most difficult to investigate. The roots of this system are restricted to a few selectively 5-HT synthesising neurons in the midbrain, the pons and the medulla oblongata, which together represent the different groups of nuclei raphe B1-B9 (A. Dahlström, K. Fuxe, Acta Physiol. Scand. 62: Suppi. 232, 1 (1964)). Moreover, 5-HT constitutes an intermediate in the biosynthesis of the hormone melatonin and therefore the epiphysis has its maximal activity in the dark phase of a 24 hour-regulation, in which it produces the highest amounts of TPH (J. M. Miguez, F. J. Martin, M. Aldegunde, Neurochem. Res. 22, 87 (1997)).

Besides its occurrence in the brain and the epiphysis, TPH was also discovered in enteric neurons (E. Fiorica-Howells, L. Maroteaux, M. D. Gershon, J. Neurosci. 20, 294 (2000)), pre-implanted embryos (D. J. Walther, M. Bader, Mol. Brain Res. 68, 55 (1999)), mast cells (L. M. Finocchiaro et al., J. Interferon Res. 8, 705 (1988)) and—most strikingly—in the enterochromaffin cells of the gastrointestinal tract (L. J. Weber, A. Horita, Biochem. Pharmacol. 14, 1141 (1965)). These cells are expected to be the source of 5-HT in the blood, where it is nearly completely stored in tight storage vesicles of the thrombocytes (J. Champier et al. Lire Sei. 60, 2191 (1997)). 5-HT is involved in different processes in the peripheral tissue, like e.g. regulation of the vascular tonus (M. M. Rapport, A. A. Green, I. H. Page, J. Biol. Chem. 176, 1237 (1948)), intestinal motility (M. D. Gershon, Aliment. Pharmacol. Ther. 13, 15 (1999)), primary haemostasis (J. M. Holland, Proc. Exp. Biol. Med. 151, 32 (1976)) and cell-mediated immune responses (G. P. Geba et al. J. Immunol. 157, 557 (1996)). Since the amount of 5-HT in the brain and the periphery is directly connected with the TPH-activity, the understanding of TPH-expression and -regulation is an important step to clarify the functions of 5-HT in the CNS and in the peripheral tissue.

### DEFICIENCY OF THE STATE OF THE ART

The presence of TPH mRNA and protein in the vertebrate CNS was reported about ten years ago, however the data were inconsistent. In the epiphysis and in the nuclei raphe (S. Dumas, M. C. Darmon, J. Delort, J. Mallet, J”. Neurosci. Res. 24, 537 (1989); R. P. Hart, R. Yang, L. A. Riley, T. L. Green, Mol. Cell. Neurose!. 2, 71 (1991)) e.g. different mRNA to protein ratios were discovered: comparable TPH-protein levels are present in the nuclei raphe and in the epiphysis, whereas the mRNA-levels are at least 1000 times higher in the latter (F. Chamas, L. Serova, E. L. Sabban, Neurose!. Lett. 267, 157 (1999)). All studies attributed these differences in the protein-mRNA-ratios to different translational efficiencies of mRNAs, which are variably spliced in the 5′-untranslated regions.

However, hints for the existence of different TPH-isoforms (C. D. Cash, Gen. Pharmac. 30, 569 (1998), 21 S. M. Mockkus, K. E. Vrana, J. Mol. Neurose!. 10, 163 (1998)) had been existent for more than 30 years in literature: a) in a purification method, two activity peaks in brain homogenates (H. Nakata, H. Fujisawa, Eur. J. Biochem. 122, 41 (1982)) were discovered and partially purified enzymes with definite biochemical properties were described in dependence of the analysed tissue (D. M. Kühn, M. A. Meyer, W. Lovenberg, Arch. Biochem. Biophys. 199, 355 (1980)), b) the first generation of antibodies against TPH, which was purified from a mouse mastocytoma cell line (P815) did not cross-react with the brain-TPH(H. Nakata, H. Fujisawa, Eur. J. Biochem. 124, 595 (1982)), although the antibody, which currently is commercially available, does cross-react. Until now, these phenomena has been attributed to different phosphorylation states of the proteins (C. D. Cash, Gen. Pharmac. 30, 569 (1998)).

A founded explanation for the mentioned differences of the mRNA- and protein levels or for the contradictory data, however, was missing. This object has been achieved in the present invention.

### CONTEXT OF OBJECTIVE AND SOLUTION

In the present specification, it is shown for the first time by means of “gene targeting”, that serotonin is independently synthesised by two different tryptophane hydroxylase isoenzymes in the peripheral tissues and in the neurones. Furthermore, a specifically neuronal isoform of tryptophane hydroxylase is identified for the first time. Although the amino acid sequences of the two tryptophane hydroxylases are highly homologous to each other, the gene sequences only display a little similarity, which explains, why the neuronal tryptophane hydroxylase (in the following designated as snTPH) remained undiscovered for decades. The isolation of the specifically neuronal expressed snTPH can be useful for the development and preparation of novel therapeutics for the treatment of neuronal/psychiatric disorders.

According to this, it is an essential objective of the present invention to provide a method, by which neuronal and psychiatric diseases can be treated by means of affecting serotonin metabolism via modifying the amount/activity of snTPH. Furthermore, it is an objective of the present invention to provide nucleic acid molecules and isoform specific inhibitors/activators, which can be employed for producing pharmaceutical preparations for the treatment and diagnosis of neuronal disorders, based on affecting the serotonin metabolism by means of snTPH. Further objectives will become obvious from the following description.

These objectives are achieved by the subjects of the independent claims, which are particularly based on providing the DNA sequences according to the invention, the gene products of which can be directly employed for affecting the neuronal serotonin metabolism. Favourable aspects are defined in the dependent claims.

In the present invention, it has—surprisingly and for the first time—been succeeded in providing nucleic acid molecules encoding for a protein with the enzymatic activity of a neuronal tryptophane hydroxylase (snTPH). To investigate the physiolgical effects of a loss of 5-HT-synthesis, mice with a genetic deficiency for TPH were generated. Although a lethal pheno-type of this genetic manipulation was expected, as it was determined for animals with a deficiency for TH (Q. Y. Zhou, C. J. Quaife, R. D. Palmiter, Nature 374, 640 (1995)), surprisingly viable homozygous TPH-knock out-mice (TPH(−/−)) were generated. These mice lack 5-HT in the periphery. It was possible to determine for the first time, that the mode of action of 5-HT in primary haemostasis is based on a release of Von-Willebrand-Factor (vWf). Completely unexpected, there was only a slight diminution of stable 5-HT amounts in the classic serotonergic brain regions of TPH(−/−) mice, leading to the identification of a TPH-isoform, which is exclusively expressed in neurones.

The present invention thus comprises a recombinant nucleic acid sequence coding for a polypeptide with neuronal tryptophane hydroxylase activity, selected from a group of:

a) a nucleic acid sequence with the sequence shown in SEQ ID No:1, SEQ ID No:3 or SEQ ID No:5 (DNA-sequence from human, mouse, rat) or fragments thereof,

b) nucleic acid sequences (such as DNA, cDNA, mRNA, PNA, siRNA, and the like), which can be deduced from the nucleic acid sequence shown in SEQ ID No:1, SEQ ID No:3 or SEQ ID No:5 as a result of the degenerated genetic code, or fragments thereof,

c) derivatives of the nucleic acid sequence shown in SEQ ID No:1, SEQ ID No:3 or SEQ ID No:5, these derivatives encoding for polypeptides according to SEQ ID No:2, SEQ ID No:4 or SEQ ID No:6 (amino acid sequence of human, mouse, rat), thereby displaying at least 80% homology at the amino acid level and being without a significant decrease in the biological activity of the polypeptides,

d) a human genomic nucleic acid sequence containing the gene for sn-TPH and displaying polymorphisms.

It has to be stated, that the wording “neuronal tryptophane hydroxylase” (sn-TPH) in the text of this specification is meant to include all peptides and proteins with tryptophane hydroxylase activity.

These nucleic acids among eukaryotic and prokaryotic organisms are preferably to be found in mammalians like Homo sapiens (human), Rattus norvegicus (rat) or Mus musculus (mouse). These nucleic acids encode for the amino acid sequences of SEQ ID No:2 (Homo sapiens), SEQ ID No:4 (Mus musculus) or SEQ ID No:6 (Rattus norvegicus). These nucleotide sequences are designated in the following as snTPH-genes and their homologues, the amino acid sequences as snTPH and their homologues.

The nucleic acid sequences according to the invention encode for the above described proteins, in particular for those with the primary structures shown in SEQ ID No:2, SEQ ID No:4 or SEQ ID No:6. The nucleic acid sequence from Homo sapiens is depicted in SEQ ID No:1, that from Mus musculus in SEQ ID No:3 and that from Rattus norvegicus in SEQ ID No:5. After isolation and sequencing, the nucleic acid sequences according to the invention according to SEQ ID No:1, SEQ ID No:3 or SEQ ID No:5 or their functional equivalents like e.g. allelic variants, are available. Allelic variants of SEQ ID No:1, SEQ ID No:3 or SEQ ID No:5 mean variants, which display 60 to 100% homology at the amino acid level (=identity), preferably 70 to 100% homology, especially preferable 90 to 100% homology. Allelic variants comprise in particular those functional variants, which are available from the sequences depicted in SEQ ID No:1, SEQ ID No:3 or SEQ ID No:5 by means of deletion, insertion or substitution of nucleotides, whereby at least one of the essential biological activities is maintained.

Homologous or sequence related nucleic acid sequences can be isolated from all mammalian species including humans by common methods, e.g. homology screening by hybridisation with a probe of the nucleic acids according to the invention or with parts thereof.

Hybridisation in the scope of this invention means a hybridisation under conventional hybridisation conditions, preferably under stringent conditions, as they are e.g. described in Sambrook et al. (Sambrook et al. 1989. Molecular Cloning: A Laboratory Manual, 2. edition, Cold Spring Harbor Laboratory Press, Cold Spring Harbor, N.Y.).

Standard hybridisation conditions e.g. mean—in dependence of the nucleic acid-temperatures between 42° C. and 58° C. in an aqueous buffer solution with a concentration of 0.1 to 5×SSC (1×SSC=0.15 M NaCl, 15 mM sodium citrate, pH 7.2) or additionally in the presence of 50% formamide like e.g. 42° C. in 5×SSC, 50% formamide. DNA-sequences hybridising to the DNA-sequences encoding for snTPH can e.g. be isolated from the genomic libraries or cDNA-libraries of any vertebrate, preferably from mammalians, which are in possession of the DNA-sequences according to the invention. Of course, the conditions have sometimes to be suitably adapted in order to distinguish between snTPHs and other TPHs. Such adaption can easily be achieved by standard experiments well known to the skilled artisan. The identification and isolation of such DNA-sequences can e.g. be accomplished by using DNA-sequences comprising exactly or nearly the DNA-sequence depicted in SEQ ID No:1, SEQ ID No:3 or SEQ ID No:5 or parts thereof, or respectively by using the reverse complementary sequences thereof for hybridisation according to standard protocols (e.g. Sambrook et al. (1989), see above). The fragments used as hybridisation probes may also be synthetic fragments produced by means of common techniques of synthesis, whereat the sequence of these fragments largely corresponds to the above mentioned sn-TPH-sequences or parts thereof.

Nucleic acid sequences encoding for a protein with the biological activity of a neuronal tryptophane hydroxylase also comprise DNA-sequences, the nucleic acid sequences of which are degenerated in relation to one of the before mentioned nucleic acid sequences. The degeneration of the genetic code offers the expert—among other aspects—the possibility to adapt the nucleotide sequence of the respective DNA-sequence to the codon preference of the target organisms, thereby optimising gene expression. The DNA-sequences described above also include fragments, derivatives and allelic variants of the above described DNA-sequences encoding for a protein with the biological activity of a neuronal tryptophane hydroxylase. The wording “fragment” in this context designates parts of the DNA-sequence, which are long enough to encode for one of the described proteins.

The wording “derivative” in this context means, that the sequences differs from the above described sequences at one or several positions but displays a high degree of homology. Homology herein denotes a sequence identity of at least 25 percent, in particular an identity of at least 40 percent, preferably of at least 60 percent, especially preferred of over 80 percent and most preferred of over 90 percent. Thereby, the proteins encoded by these DNA-sequences display a sequence identity to the amino acid sequences given in SEQ ID No:2, SEQ ID No:4 or SEQ ID No:6 of at least 60 percent, in particular of at least 80 percent, preferably of 85 percent and especially preferred of over 90 percent, 95 percent and 98 percent. The deviations to the above described DNA-sequences may be due to deletion, substitution, insertion or recombination. The nucleic acid sequences according to the invention or their fragments may moreover be employed for the isolation of genomic sequences by means of homology screening using the above mentioned hybridisation conditions.

A further subject of the invention are the isolated proteins according to the invention. These constitute the proteins comprising an amino acid sequence depicted in SEQ ID No:2, SEQ ID No:4 or SEQ ID No:6 or a sequence, that can be derived therefrom by substitution, inversion, insertion or deletion of one or several amino acid residues, provided that at least one essential biological property of the proteins according to SEQ ID No:2, SEQ ID No:4, SEQ ID No:6 is preserved. Thereby, e.g. specific amino acids may be substituted by amino acids with similar physico-chemical properties (spacial dimensions, basic character, hydrophobic character, etc.). Additionally, one or several amino acids may be exchanged with regard to their position or may be added, deleted or changed by a combination of these measures. The proteins being such modified in respect to SEQ ID No:2, SEQ ID No:4 or SEQ ID No:6 display at least 60%, preferably 70% and especially preferred at least 90% of sequence identity in relation to the sequences of SEQ ID No:2, SEQ ID No:4 or SEQ ID No:6, whereat the sequence identity is determined according to the algorithm of Altschul et al. (1990, J. Mol. Biol., 215: 403-410, BLAST-programme (current version), determined over the entire length of the proteine sequences).

The wording “functional equivalents” of the mentioned sequences designates nucleic acids encoding for proteins possessing the biological activity of snTPH as described herein, and at least a 50% activity compared to the sequences shown in SEQ ID No:2, SEQ ID No:4 or SEQ ID No:6. These functional equivalents preferably interact with other proteins by forming so-called protein complexes (protein heteromers) therewith.

In a further embodiment, also the specific binding of synthetic or natural agonists and antagonists to the proteins according to the invention (the proteins possessing the amino acid sequences of SEQ ID No:2, SEQ ID No:4 or SEQ ID No:6 or their functional equivalents) constitutes a part of the proteins' essential biological properties, which can be altered or modified in consequence of this binding behaviour.

The proteins according to the invention and their functional variants are advantageously isolated from the brain of mammalian species like Homo sapiens (human SCLC-cells, SHP-77 and other examples), Mus musculus or Rattus norvegicus. Moreover, the invention refers to nucleic acid molecules, which comprise the nucleic acid sequences according to the invention or which have been produced or deduced from these sequences by means of naturally occurring or genetic or chemical processes or synthesis methods. Possible examples for these molecules are DNA- or RNA-molecules, cDNA, genomic DNA, mRNA, etc.

It is of further advantage to functionally combine the nucleic acids according to the invention with at least one genetic regulatory element in order to generate the recombinant nucleic acid molecules according to the invention (expression cassette, gene construct), which enable transcription and—if desired—translation within a cell. To this aim, the nucleic acid sequences according to the invention are in general functionally connected to genetic regulatory elements like transcriptional or translational signals. Thereby, the nucleic acid sequence may be connected in the antisense- or sense-direction to one or several regulatory signals. According to the intended application, this combination may lead to an increased or decreased gene expression.

In principle, all naturally occurring promoters and their regulatory sequences are suitable for expressing the DNA-sequences comprised within the recombinant nucleic acid molecules according to the invention in eukaryotic or prokaryotic cells.

The regulatory sequences or factors may thereby preferably affect the expression in a positive manner leading to an increase of expression. An enforcement of the regulatory elements can e.g. be realised advantageously at the level of transcription by providing strong transcriptional signals like promotors. Another favourable possibility is a targeted genetic modification of the promotor occurring naturally upstream of the respective sequences. Additional regulatory signals like enhancers or polyadenylation signals or terminators positioned at the 3′ site may be functionally employed in the recombinant nucleic acid molecules.

In any case, the expert can find suitable promoters in literature or can isolate them from arbitrary organisms by means of standard methods.

Furthermore, transcriptional or terminating sequences are present, which may serve to properly terminate transcription or to add a poly(A)-tail to the transcript, which is believed to have a function in stabilising the transcript. Elements of this kind have been described in literature and can be arbitrarily exchanged.

Moreover, the invention refers to the provision of vectors comprising the nucleic acid sequences according to the invention or a recombinant nucleic acid molecule according to the invention, the employment of which serves to create transgenic organisms with increased or decreased production/activity of snTPH.

The present invention thus also refers to vectors, in particular to plasmids, cosmids, viruses, bacteriophages and other vectors commonly used in genetic engineering, whereby these vectors comprise the before mentioned nucleic acid molecules according to the invention and can—if desired—be used for transferring the nucleic acid molecules according to the invention to eukaryotic or prokaryotic cells.

Moreover, the recombinant nucleic acid molecule or the nucleic acids according to the invention may also be expressed in the form of fragments, which are suited for therapeutic or diagnostic applications. In order to generate the recombinant proteins, vector systems or oligonucleotides can be employed, which elongate the nucleic acid or the recombinant nucleic acid molecule by adding specific nucleotide sequences, thereby encoding for modified polypeptides, the purification of which is thereby facilitated (addition of TAGs).

As a host organism, in principle all organisms are suitable, which allow an expression of the nucleic acids according to the invention, the allelic variants thereof, the homologues thereof, the functional equivalents or derivatives thereof, the recombinant nucleic acid molecule or the vectors. The wording “host organism” is to be understood as including e.g. eukaryotic or prokaryotic host organisms like bacteria, fungi, yeasts, plant cells or animal cells. Preferred organisms are bacteria like Escherichia coli, Streptomyces, Bacillus or Pseudomonas, eukaryotic microorganisms like Saccharomyces cerevisiae, Aspergillus, higher eukaryotic cells from human or animals like e.g. the cell lines COS7, NG108-15 and P815.

These transgenic or recombinant organisms are characterised by the feature, that they either as natural organisms—do not contain the nucleic acids according to the invention, the allelic variants thereof, the homologues thereof, the functional equivalents or derivatives, the recombinant nucleic acid molecule or the vectors; or that they do not contain these elements at this site in the genome or in the cell; or that the natural promoter of the recombinant nucleic acid molecules according to the invention was genetically modified such, that the respective genes are accordingly expressed in an increased or decreased manner.

Thereby, the described host organism can contain at least one nucleic acid sequence according to the invention or at least one recombinant nucleic acid molecule or at least one vector according to the invention.

Preferably, the gene product can also be expressed in transgenic organisms like transgenic animals, e.g. in mice, rats, cheeps, bovine animals or pigs. Also transgenic plants are contemplated, whereat proteins may be yielded as a result of molecular farming. In the case of transgenic organisms, also the so-called “knock-out-animals” are contemplated.

Thereby, the transgenic animals may contain a functional or non-functional nucleic acid sequence according to the invention or a functional or non-functional nucleic acid construct.

A further aspect of the present invention is the use of nucleic acids according to the invention or of parts thereof for the purpose of genetic therapy in mammals, e.g. in humans. Also sequences, which are complementary to the nucleic acids according to the invention or to parts thereof, can be employed for genetic therapy. Genetic therapy thereby comprises all therapeutic methods, which either introduce sequences according to the present invention into the body or parts of the body or which affect the expression of sequences according to the present invention. To this aim, one can employ oligonucleotides like e.g. antisense-oligonucleotides or RNA-DNA hybrid oligonucleotides with arbitrary modifications, which comprise parts of the sequences according to the present invention. Also viral constructs containing a sequence according to the present invention or parts thereof can be employed.

A further advantageous embodiment is the use of the nucleic acid sequences according to the invention, the recombinant nucleic acid molecules according to the invention or the amino acid sequence according to the invention for the identification, discovery or detection of proteins, which exhibit specific binding affinities for a protein characterised by the polypeptide (i.e. amino acid sequence) according to the present invention or for the identification of nucleic acids encoding for proteins with specific binding affinities for amino acid sequences according to the present invention. In a preferred embodiment, the Two-Hybrid System or other biochemical methods may be used for this purpose either alone or in combination. Thereby, interaction domains of the proteins according to the invention and other conserved regions and thus potential pharmacological targets can be identified.

Thus, a further aspect of the invention is the use of the Two-Hybrid System or of other biochemical methods alone or in combination for identifying the interaction domains of snTPH and the application for pharmacotherapeutic use.

A further advantageous embodiment of the invention is the use of a nucleic acid sequence according to the present invention as a marker for human hereditary diseases, in particular neuronal diseases.

A further aspect of the present invention is the use of a protein according to the present invention, the functional equivalents thereof or the peptide fragments thereof as an antigen for producing specific polyclonal or monoclonal antibodies or antibody mixtures directed against proteins according to the present invention. The wording “antibody” thereby comprises polyclonal, monoclonal, human or humanised or recombinant antibodies or fragments thereof, single chain antibodies and also synthetic antibodies. The antibodies according to the invention or their fragments in principle refer to all immune globulin classes or their subclasses or their mixtures. As examples for fragments, all truncated or modified antibody fragments with one or two binding sites complementary to the antigen are to be mentioned. Also genetically modified non-truncated fragments may be advantageously employed. The antibodies or fragments can be used alone or in mixtures. The antibody genes for genetic procedures can e.g. be isolated from the hybridoma cells in a way familiar to the expert. To this aim, antibody-producing cells are grown and, after the cells have reached a sufficient optical density, the mRNA is isolated from the cells in a well known manner by cell lysis with guanidine thiocyanate, acidification with sodium acetate, extraction with phenole, chloroform/iso-amyl alcohol, precipitation with isopropanol and washing with ethanol, followed by the synthesis of cDNA from the mRNA by means of reverse transcriptase. The synthesised cDNA may be directly used for vector insertion or expression or modified by genetic manipulation, e.g. by site directed mutagenesis, introduction of insertions, inversions, deletions or nucleotide base exchanges, followed by an introduction into suitable animal, fungal, bacterial or viral vectors and an expression in the respective host organisms.

Specific antibodies against the proteins according to the invention can be suitable both as diagnostic agents and as therapeutics in disease conditions, which are—among other features characterised by changes in the serotonin metabolism.

The nucleic acid sequences according to the invention according to the present invention, their functional equivalents, homologues, derivatives or fragments, the proteins encoded by them and comprising the amino acid sequences according to the invention as well as the reagents derived therefrom (oligonucleotides, antibodies, peptides) can be used for detecting a predisposition, for diagnosis, for therapy and for monitoring of neurological diseases, in particular in the context of serotonergic effects in behavioural physiology.

Affecting the snTPH can be accomplished in the following ways:

Specific inhibitors of neuronal TPH-isoforms are developed, which are based on the specific molecular differences of the TPH-isoforms. Also developed are inhibitors, which are not permeable for the blood-brain barrier. Specific inhibitors for the TPH-isoforms can be identified in vitro by standard screening methods familiar to the expert, since the identified cDNAs of snTPH can be used in expression systems in order to specifically express the pure isoforms.

These inhibitors—besides their diagnostic benefits—also offer therapeutic applications, since increased serotonin levels in the CNS are found in a plurality of medical complications.

Another aspect of the present invention therefore relates to a method for isolating a compound that binds to a polypeptide according to the present invention, comprising the steps of: (a) contacting a mammalian cell which expresses a polypeptide according to the present invention having sn-TPH activity with a compound; (b) detecting the presence of the compound which binds to the sn-TPH polypeptide, and (c) determining, whether the compound binds said sn-TPH polypeptide. Such so-called “binding-assays” are standard practice in the art and can include suitably labelled, e.g. radioactively labelled, dye-labelled, enzyme labelled, antigen labelled and/or mass-tag labelled putative binding compounds that can be detected using the respective suitable agent, e.g. antibodies, enzyme substrates, and the like.

Yet another aspect of the present invention relates therefore to a method for the production of a pharmaceutical composition comprising the steps of the method as given above and the subsequent step of formulating the compound identified in step (c) and/or its pharmaceutically acceptable salts in a pharmaceutically acceptable form. Suitable formulations depend inter alia from the route of administration and can easily be produced by the person of skill, based on the information as given in the respective literature. These pharmaceutical compositions may be administered orally, rectally, parenterally, intracistemally, intravaginally, intraperitoneally, topically (as by powders, ointments, drops or transdermal patch), bucally, or as an oral or nasal spray.

As used herein, “pharmaceutically acceptable carrier” is intended to mean a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. One of ordinary skill will recognise that the choice of a particular mode of administration can be made empirically based upon considerations such as the particular disease state being treated; the type and degree of the response to be achieved; the specific agent or composition employed; the age, body weight, general health, sex and diet of the patient; the time of administration and rate of excretion of the agent or composition; the duration of the treatment; drugs (such as a chemotherapeutic agent) used in combination or coincidental with the specific composition; and like factors well known in the medical arts.

Pharmaceutical compositions of the present invention for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Illustrative examples of suitable aqueous and nonaqueous carriers, diluents, solvents or vehicles include, but are not limited to, water, ethanol, polyols (such as glycerol, propylene glycol, polyethylene glycol, and the like), carboxymethylcelulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminium monostearate and gelatine.

Affecting the snTPH-regulation (activity and/or amount of snTPH) may be accomplished in the following ways according to the invention:

A specific down-regulation of snTPH can be accomplished biochemically by means of ribozymes, antisense-oligonucleotides or by means of antisense-RNA expression, whereat the sequence differences of the isoform mRNAs allow to influence just one mRNA. Furthermore, also pharmacological approaches based on specific TPH-inhibitors like e.g. p-chlorophenylalanine or p-ethinylphenylalanine can be employed according to the invention.

The serotonin production is stimulated in a molecular biological manner preferably by tissue-specific overexpression of snTPH. In the pharmacological approach, both the precursor substance, 5-hydroxy-tryptophane and substituted analogues can be applied, as well as serotonin itself.

The method for diagnosing neuronal diseases according to the invention is characterised thereby, that a specific inhibition of the peripheral serotonin biosynthesis is accomplished, which is followed by measuring the metabolite concentration stemming from the CNS and by determining the severity of the disease by means of a comparative graph. To this aim, it is necessary to employ substances, which cannot cross the blood-brain barrier.

Another aspect of the present invention is related to a method for determining the pharmacogenetic properties of a pharmaceutically active compound, comprising a) administering the compound to a mammal, b) determining the level of expression of snTPH in a biological sample obtained from said mammal, and c) comparing said level of expression of snTPH with a level obtained from a control sample. A “pharmaceutically active compound” according to the present invention is a compound that alleviates the symptoms of a medical condition related to the level of expression or the function of snTPH, either alone or in combination with other compounds, such as, for example, used in the combination therapeutic as described herein. The pharmaceutically active compound can be derived from so-called “small molecules” (i.e. having a molecular weight of less than 500 kDa) or larger molecules, such as peptides, proteins, antibodies, oligonucleotides, and the like.

Yet another aspect of the present invention relates to a method for the improved treatment of a disease, comprising performing the method as described above, and increasing or decreasing the doses of the pharmaceutically active compound to be applied to said patient. The inclusion of specifically derived pharmacogenetic information in the treatment of a patient is a particular aspect of the so-called “personalised-medicine”. Personalised treatment proves to be more efficient than the general “standard” application of a doses of a medicament (i.e. containing a pharmaceutically active compound) (see, for example, Kalow W. Pharmacogenetics and personalised medicine. Fundam Clin Pharmacol. 2002 October;16(5):337-42; Jain K K. Personalised medicine. Curr Opin Mol Ther. 2002 December;4(6):548-58; Ross J S, Ginsburg G S. Integrating diagnostics and therapeutics: revolutionising drug discovery and patient care. Drug Discov Today. 2002 Aug. 15;7(16):859-64; Ross J S, Ginsburg G S. The integration of molecular diagnostics with therapeutics. Implications for drug development and pathology practice. Am J Clin Pathol. 2003 January;119(1):26-36; Ikeda M, Kitajima T, Iwata N, Ozaki N. Molecular genetics of mood disorders, Nihon Shinkei Seishin Yakurigaku Zasshi. 2002 October;22(5):137-43; Pagliarulo V, Datar R H, Cote R J. Role of genetic and expression profiling in pharmacogenomics: the changing face of patient management. Curr Issues Mol. Biol. 2002 October;4(4): 101-10; Iida A, Saito S, Sekine A, Nakamura Y. SNP collection, pharmacogenomics, and the future of drug therapy Gan To Kagaku Ryoho. 2002 September;29(9):1665-73; Smith D J, Lusis A J. The allelic structure of common disease. Hum Mol Genet. 2002 Oct. 1;11(20):2455-61.).

The above-indicated methods can be used in the diagnosis and/or treatment of diseases that are selected from neuronal diseases, such as sleep disturbances, anxiety, alcoholism, drug abuse, disorders of food uptake and/or sexual disorders.

Furthermore, it is advantageous to use the nucleic acid sequences according to the invention, the recombinant nucleic acid molecules according to the invention or the amino acid sequences according to the invention for the treatment of sleep disturbances, anxiety, alcoholism, drug abuse, abnormal food intake or sexual disturbances, characterised in that the serotonin level is modified by modulating the gene expression of snTPH or by modifying the snTPH activity.

In a further embodiment, the nucleic acid sequences according to the invention, the recombinant nucleic acid molecules according to the invention or the amino acid sequences according to the invention are used for the development and production of a combination therapeutic, which comprises a protein according to the present invention and at least one further protein, in particular for the regulation of the serotonin metabolism. Besides a common carrier material, the combination therapeutic can in principle comprise each pharmaceutically well-tolerated protein. In a preferred embodiment, the combination therapeutic as a further active substance contains a peripheral tryptophane hydroxylase for the simultaneous treatment of neuronal/psychiatric diseases and thrombotic diseases like coronary infarction and stroke (the peripheral and the neuronal production of serotonin are simultaneously affected in the form of an increase or decrease). In this context, it should be mentioned, that the use of SSRI (serotonin specific reuptake inhibitor)-antidepressants in psycho-pharmacological therapy may in some cases lead to severe bleeding complications. This can be prevented by the employment of a preparation with specific neuronal effect. Accordingly, a further embodiment of the invention is the described combination therapeutic for the treatment of bleeding episodes in the psycho-pharmacological treatment of depressions with antidepressants affecting the serotonin reuptake transporter, comprising antidepressants and von Willebrand-factor.

### EXAMPLES

The described invention will be further explained hereafter by means of the following examples.

The expert will recognise various other embodiments from the present description. It is thus explicitly stated, that the examples, the description of the figures and the figures merely have the purpose of elucidation and are not to be understood as restrictions of the invention.

In case of not being otherwise indicated, the experimental procedure was accomplished according to the protocols of Ausubel et al. (eds.), 1998, Current Protocols in Molecular Biology. John Wiley & Sons, New York and Sambrook et al. (eds.), 1989, Molecular cloning: A laboratory manual. CSH Laboratory Press, New York.

Molecular Biological Methods and Generation of Tph−/−Mice.

All cloning procedures, PCR, and Southern blotting were conducted according to standard protocols (J. Sambrook, E. F. Fritsch, T. Maniatis, Molecular Cloning: A Laboratory Manual. 2nd edn.: 1 pp (1989)). For the generation of a targeting vector, a 2.2 kb and a 8.6 kb amplicon were obtained with long range PCR using primers derived from the published Tph sequence of the mouse (J. Stoll, C. A. Kozak, D. Goldman, Genomics 7, 88 (1990)) (5′-GACATCGGATCAGAAGACTCCC-3′ (SEQ ID No. 7)/5′-CTCCCTCTTTCGGAGGAATGG-3′ (SEQ ID No. 8), and 5′-CACCATGATTGAAGACAACAAGG-3′ (SEQ ID No. 9)/5′-CGTGAATTCAATCTTGGGAATGG-3′ (SEQ ID No. 10), respectively). The 2.2 kb fragment was cloned into pCRII-T-vector (Invitrogen, Leek, NL) to obtain pInlc2, and subsequently sequenced. As short arm of the targeting vector, a 1 kb Fragment was isolated by Hind III digestion and T4-DNA polymerase blunting, followed by Xho I digestion. To obtain a long arm for the targeting vector, the 8.6 kb fragment was first partially digested with BamH I, and then digested with Spe I, and the resulting 5.2 kb fragment was isolated. The 1 kb and the 5.2 kb fragments were then cloned into a vector containing a neomycin-resistance cassette and a TK-cassette. ES-cell targeting with this vector and embryo manipulations were carried out according to standard protocols (T. Walther et al. J. Biol. Chem. 273, 11867 (1998)). Southern blot of Eco RI-digested genomic DNA using either a internal or a external probe detects a 10 kb Eco RI fragment in wild-type animals, whereas in KO-animals a shorter 8 kb fragment is detected due to the Eco RI site contained in the neomycin resistance cassette.

Total RNA preparation and ribonuclease protection assays (RPA) were performed using TRIZOL reagent (Gibco, Eggenheim, Germany) and the RPAII kit (Ambion, Austin, IX) as previously described (D. J. Walther, M. Bader, Mol. Brain Res. 68, 55 (1999)). Radioactivity was visualized by a BAS2000 phosphoimager system (Fuji, Tokyo, Japan). For Tph1 we used a previously described RPA probe (D. J. Walther, M. Bader, Mol. Brain Res. 68, 55 (1999)). Furthermore, we generated a RPA probe for Tph2 with 5′-GGTTCCCTCGGAAGATCTCTGAGTTAGACA-3′ sense (SEQ ID No. 11) and 5′-AGAGCTCCCGGAATACAACACCCCAAGT-3′ antisense (SEQ ID No. 12) primers corresponding to parts of Tph2 exons 4 and 6, respectively.

For expression studies, we amplified the complete coding sequence with 5′-TGCTCTTCAGCACCAGGGTTCTGGAC-3′ sense (SEQ ID No. 13) and 5′-AGAATTGCATGCTTACTAGCCAACC-3′ antisense (SEQ ID No. 14) primers and cloned the cDNA into a eukaryotic expression vector under control of a CMV promotor. We transiently transfected COS7 cells with this vector using the Ca₃(PO₄)₂ coprecipitation method (J. Sambrook, E. F. Fritsch, T. Maniatis, Molecular Cloning: A Laboratory Manual. 2nd edn.: 1 pp (1989)). Cell culture was conducted as described (D. J. Walther, J.-U. Peter, M. Bader, Cancer 94, 3135 (2002)).

TPH Activity Assay.

The activity of cell homogenates was determined using an adapted method basically as described (E. M. Gal, K. Patterson, Analyt. Biochem. 52, 625 (1973)), monitoring for 5-HTP accumulation by HPLC in presence of NSD1015, a inhibitor of aromatic amino acid decarboxylases, in the reaction mixture. Briefly: cells were harvested with a scraper and washed twice with phosphate buffered saline, resuspended in 75 mM tris-acetate buffer (pH 7.5), and lysed by sonication. After withdrawal of an aliquot for protein determination, the homogenates were immediately pre-incubated in 100 μl buffer containing 2 mg/ml catalase, 25 mM DTT and 100 μM Fe(NH4)2(SO4)2 for 10 min at 30° C. in the dark. The pre-incubated samples were incubated at 37° C. for 30 min after addition of 400 μl 15 mM tris-acetate buffer (pH 6.4) containing a final concentration of 300 μM L-Trp and 300 μM 6-methyltetrahydropterin and 2 mM NSD1015. The reaction was terminated by addition of perchloric acid to a final concentration of 300 mM. All reagents were purchased from Sigma, Deisenhofen, Germany.

Analytical HPLC Methods.

The brains were rapidly removed, immediately frozen on dry ice and stored at −80° C. until use. The hippocampus and frontal cortex were dissected from the frozen brain on a cold plate (−10° C.) according to Paxinos and Watson (G. Paxinos, C. Watson, The Rat Brain In Stereotactic Coordinates. Academic Press, Inc., NY, 1 (1997)). Tissue extracts for the determination of 5-HT, Trp, and 5-HIAA contents were obtained and analysed using reverse phase HPLC with fluorometric detection (HPLC-FD) as previously described (D. J. Walther, M. Bader, Mol. Brain Res. 68, 55 (1999)), or analysed using a HPLC coupled with electrochemical detection (HPLCECD; Decade, Antec Leyden B V, Leiden, The Netherlands) as previously described (G. Sperk, J. Neurochem. 38, 840 (1982)).

Behavioural Studies.

All behavioural studies were conducted as previously described (T. Walther et al. J. Biol. Chem. 273, 11867 (1998), H. Thomas, H. Fink, T. R. Sohr, M. Voits, Pharmacol. Biochem. Behav. 65, 15 (2000), P. Gerhardt, M. Voits, H. Fink, J. P. Huston, Peptides 15, 689 (1994), R. U. Hasenöhrl, C. Frisch, S. Nikolaus, J. P. Huston, Behav. Neural Biol. 62, 110 (1994)) using 11 to 12 animals of each genotype.

TABLE 1
Behavioural parameters of Tph−/− mice.

| Elevated Plus Maze | | | |
|---|---|---|---|
| (10 min) | Tph+/+ (n = 11) | Tph−/− (n = 12) | |
| total distance | 5.5 ± 0.6 | 5.8 ± 0.4 | (n.s.) |
| travelled (m) | | | |
| total no. of entries | 27.8 ± 2.9 | 21.9 ± 3.5 | (n.s.) |
| % entries open of | 18.3 ± 2.6 | 19.0 ± 2.4 | (n.s.) |
| total no. of entries | | | |
| % time open of total | 5.6 ± 2.0 | 4.2 ± 0.8 | (n.s.) |
| time in zones | | | |

| Hole Board (10 min) | Tph+/+ (n = 12) | Tph−/− (n = 12) | |
|---|---|---|---|
| movement time (sec) | | | |
| day 1 | 388.8 ± 20.2 | 392.7 ± 19.8 | (n.s.) |
| day 2 | 289.3 ± 32.2* | 304.8 ± 19.6* | (n.s.) |
| distance (cm) | | | |
| day 1 | 797.2 ± 75.7 | 883.2 ± 78.8 | (n.s.) |
| day 2 | 52.2.6 ± 93.9* | 581.5 ± 63.0* | (n.s.) |
| no. of nosepokes | | | |
| day 1 | 8.0 ± 1.3 | 6.3 ± 0.8 | (n.s.) |
| day 2 | 3.6 ± 0.9* | 1.9 ± 0.5* | (n.s.) |
| nosepokes - | | | |
| cumulative time | | | |
| day 1 | 9.8 ± 1.7 | 8.1 ± 1.1 | (n.s.) |
| day 2 | 4.0 ± 1.0* | 2.8 ± 0.7* | (n.s.) |

Numbers are the mean values ± SEM;
n.s.: not significant;
*p < 0.05 vs. day 1.

GenBank Accession Numbers. The following Tph sequences can be found in databases: Chicken Tph1 P70080, chicken Tph2 (partial) EST AL584678. Zebrafish Tph1 (partial) EST BI475520, zebrafish Tph2 (partial) EST BI979362. Mouse Tph1 P17532, mouse Tph2 new entry AY090565. Rat Tph1 P09810, rat Tph2 new entry AY098915. Human Tph1 P17752, human Tph2 new entry AY098914.

Unchanged 5-HT Biosynthesis in the CNS of TPH(−/−)-Mice

A “gene targeting”-vector was created, which substituted the 3′-coding region of the first translated exon of the tph-gene by a transcriptionally active gene cassette conferring a neomycin resistance in order to inactivate TPH-expression (FIG. 1A). Mice with a TPH-deficiency were generated by means of standard protocols for gene targeting (Walther et al., J. Biol. Chem. 273, 11867 (1998)). Animals being heterozygous or homozygous for the mutated tph-allele were identified by PCR (FIG. 1B) and Southern Blots with an outer (FIG. 1C) and an inner probe, which detected identical fragments.

The loss of TPH-activity in “gene targeted”-mice was first demonstrated in whole blood samples by using high pressure liquid chromatography with a fluorimetric (HPLC-FD; FIG. 1D) or electrochemical (HPLC-ECD; FIGS. 1D and E) detection. The wild type TPH(+/+)-offspring of one pair or mice parents and the inbred lines C57BL/6 and 129SvJ, of which the TPH(−/−)-animals descended, contained the expected high concentrations of 5-HT, whereas in TPH(−/−)-animals no 5-HT was detectable with these sensitive methods. Since the minimum detection limit in whole blood was 5 pg/U, the 5-HT must have been less than 1% of the normal concentration in mouse thrombocytes.

Moreover, gastrointestinal enterochromaffin cells, which constitute the source of the large 5-HT pools in whole blood, were free of 5-HT in TPH(−/−)-mice, as it was shown by immunohistochemistry (FIG. 1F), whereas distinct traces of 5-HT (less than 4% of the normal level) were surprisingly found in the duodenum of TPH(−/−)-mice when using HPLC for detection (FIG. 1D).

It is extremely remarkable, that only a little decrease of 5-HT-amounts and its most important metabolite 5-hydroxyindole-3-acetic acid (5-HIAA) were detected in the serotonergic projection areas of TPH(−/−)-mice, whereat this decrease in the hippocampus only reached statistical significance in comparison to TPH(+/+)-mice (FIG. 1E). The differences of 5-HT amounts between two laboratory mice races (C57/BL/6 and 129SvJ) were larger than the differences of 5-HT amounts found in the analysed brain regions of TPH(−/−)- and TPH(+/+)-mice. As a striking result however, the epiphysis of TPH(−/−)-mice contained less than 1% of the 5-HT and 5-HIAA found in TPH(+/+)-mice (FIG. 1E).

The presence of 5-HT and TPH in the CNS was further analysed on a qualitative level by immunohistochemical methods. In the nuclei raphe (FIG. 1G) only minimal differences in 5-HT amounts (FIG. 1H) and TPH-expression (FIG. 1) were detectable. In consequence, the targeting of the tph-gene mirrored an unexpected separation between the 5-HT-biosynthesis in the CNS and in the peripheral tissue.

In order to analyse the effects of the marginally altered 5-HT-amounts, behavioural experiments were performed with TPH(−/−)-mice and control mice. When using the “Elevated-plusmaze”-, the “Hole board”—and the “Water-maze”-tests, which indicate a behaviour associated with 5-HT, no significant differences between the behaviour of the animals with the different genotypes were detected.

Identification of a Neuronal TPH

Starting from the findings described in example 1, the reason for the maintained TPH-expression in the brain of the TPH(−/−)-animals was investigated. The existence of a second, not yet discovered genetic locus encoding for a neurospecific TPH-isoform was presumed, since the immunohistochemical experiments (FIG. 2) and the Western Blots (data not shown) indicated the presence of a TPH-protein in the brain of the TPH(−/−)-animals, which had a weight similar to the known TPH.

Upon screening the High Throughput Genomic Sequences (HTGS) of the database GenBank by using short translated TPH-sequences, a human genomic clone derived from chromosome 12 (GenBank accession: AC023966) was identified. This clone contained an open reading frame, which is very similar to the TPH exon 4, but different to PAH, which is also located on chromosome 12. A further analysis of the sequence revealed the presence of other putative exons. Moreover, a genomic rat clone (AC099197) was identified, which also contained the putative exons. Primers were developed, which are based on parts of these sequences, which are homologous to the exons 4 and 6 of mouse-TPH. These primers led to a cDNA-fragment derived from the brain of TPH(−/−)-mice, whereat this cDNA-fragment differed from known mouse forms of TPH, PAH and TH. Starting from the sequence of this fragment, the full-length cDNA of neuronal TPH was determined by using 5′- and 3′-RACE (novel GenBank entry for the neuronal TPH: AY090565). The derived amino acid sequence of this enzyme shows an homology of 86% in relation to the classical mouse-TPH sequence (FIG. 2A).

This cDNA was cloned into a eukaryotic expression vector and COSV-cells lacking an endogenous TPH activity were transiently transfected. Homogenates of transfected cells show a tryptophane hydroxylase activity confirming the specificity of this brain enzyme (FIG. 2B).

By means of RT-PCR, it was possible to confirm the expression of this second TPH-isoform in rats (novel GenBank entry: AY098915) and to identify a homologous EST-sequence in chickens (AL584678), which is different from known chicken-TPH (GGU26428). The RT-PCR analysis also revealed EST-sequences from the zebra fish brain (BI475520, B1981676 and BM025115) besides a further EST-sequence from the zebra fish ovary (BI979302), the latter displaying a high homology to known TPH. Thus, a cell-specific and mutually exclusive expression of these two TPH-isoforms seems to occur in vertebrates. Moreover, also the human homologue of the new TPH-isoform (novel GenBank entry: AY098914) was cloned and the sequence determined.

The notorious TPH mRNA was detected in the duodenum and also in the thymus gland and in the spleen, but not in the brain, when 20 μg total RNA were used in RNase protection assays being specific for the two TPH-isoforms (FIG. 2C). In contrast, the neuronal TPH is only detectable in the brain, where it is strongly expressed (FIG. 2C). Therefore, the classical TPH is designated as peripheral TPH (pTPH) and the novel enzyme as neuronal TPH (snTPH).

Furthermore, also the analysis of P815 mRNA supports a mutually exclusive cell-specific expression of snTPH and pTPH, since only pTPH mRNA, but not snTPH mRNA was successfully determined by specific RNase protection essays (RPA, FIG. 2D), and it was additionally found, why anti-TPH anti-sera produced against P815 TPH were not able to detect the enzyme derived from the brain stem. According to the mutually exclusive, cell-specific expression, it is shown in this specification, that the amounts of snTPH mRNA are not affected in the brain of the TPH(−/−)-mice (FIG. 2E). Moreover, the mid-brain and the pons—in all RNA-samples of control animals used in the RPAs—exhibited an amount of snTPH mRNA about 50 times greater than that of pTPH mRNA (FIG. 2F).

The results obtained from TPH(−/−)-mice thus show, that 5-HT is synthesised in the CNS and in the peripheral tissue by two independently controlled serotonergic systems, defined by two TPH-isoforms, which are encoded by different genes with tissue-specific and mutually exclusive expression patterns, thus readily explaining a great amount of the mysterious former data.

### DESCRIPTION OF THE SEQUENCES AND FIGURES

SEQ ID No:1: The c-DNA nucleic acid sequence of neuronal typtophane hydroxylase from Homo sapiens,

SEQ ID No:2: The amino acid sequence of neuronal tryptophane hydroxylase from Homo sapiens,

SEQ ID No:3: The c-DNA nucleic acid sequence of neuronal tryptophane hydroxylase from Mus musculus,

SEQ ID No:4: The amino acid sequence of neuronal tryptophane hydroxylase from Mus musculus,

SEQ ID No:5: The c-DNA nucleic acid sequence of neuronal tryptophane hydroxylase from Rattus norvegicus,

SEQ ID No:6: The amino acid sequence of neuronal tryptophane hydroxylase from Rattus norvegicus,

SEQ ID No:7 to SEQ ID No:10: Long range PCR primers as employed in the examples,

SEQ ID No:11 to SEQ ID No:12: RPA probe primers for Tph2 as employed in the examples, and

SEQ ID No:13 to SEQ ID No:14: sense and antisense primers as employed in the examples.

FIG. 1:

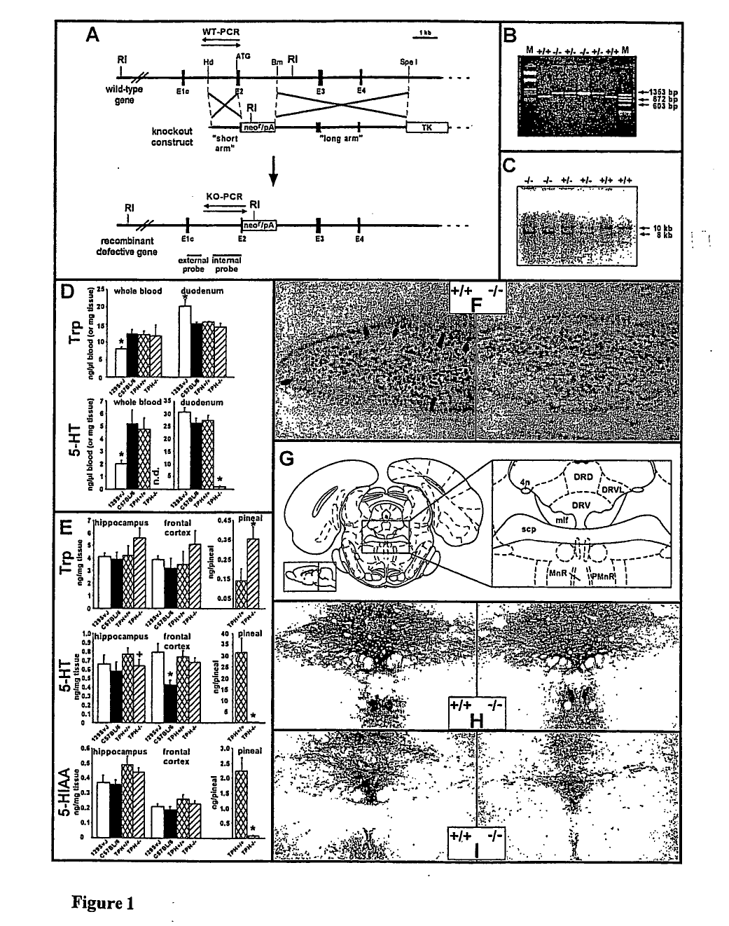

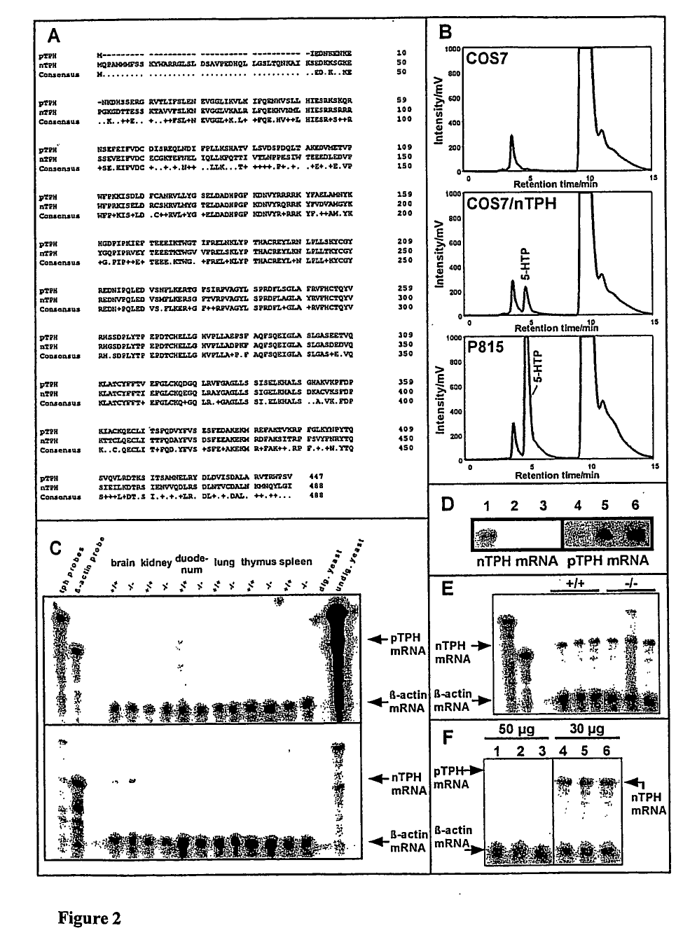

Generation of TPH(−/−)-mice; quantitative analysis of Trp and 5-HT in the peripheral tissue and quantitative analysis of Trp, 5-HT and 5-HIAA in selected brain regions using HPLC-FD and immunohistochemical staining.

(A) Schematic presentation of the targeting procedure with an indication of the first 4 of 11 exons of the TPH-gene. The knock out-construct (KO) was integrated in one allele of ES-cells by means of homologous recombination, leading to a destruction of the first coding exon (exon 2) of the TPH-gene. Furthermore, the integrated neomycin-resistance cassette (neo′) also comprises a transcriptional stop signal (pA). The positions of the analytical PCR-amplicons for identifying the wildtype and the KO-allele are indicated, as well as the positions of inner and outer probes and the EcoRI-sites (boldface: R1), which were used for the Southern Blot. Restriction sites: R1: EcoRI; Hd: HindIII; Bm: BamHI; Sc: SacI.

(B) Agarose gel electrophoresis of analytical PCR-products. As indicated in (A), the PCR experiments detect a 1.1 kb-Fragment in wildtype animals, whereas the KO-allele is detected as a 1.3 kb-fragment.

(C) Southern Blot of EcoRI-digested genomic DNA. As indicated in (A), both probes detect an EcoRI-fragment of 10 kb in the wildtype animals, whereas—due to the EcoRI-site in the neomycin resistance cassette—a shorter 8 kb-fragment is detected in the KO-animals.

(D) Trp and 5-HT in whole blood and the duodenum of the following mice lines and mutants: 129vJ, C57BL/6, TPH(−/−) and TPH(+/+). n.d.: below the minimal detection limit (<5 pg/μl); *: statistically significant (p<0.05), compared to all the other analysed mouse lines.

(E) Trp, 5-HT and 5-HIAA in the hippocampus and the frontal cortex, two serotonergic projection areas of 129vJ, C57BL/6, TPH(−/−) and TPH(+/+)-mice and in the epiphysis of TPH(−/−)-mice and TPH(+/+)-mice. *: statistically significant (p<0.05) compared to all other investigated mouse lines; +: statistically significant (p<0.05) compared to TPH(+/+)-mice, but not to other laboratory mouse lines.

(F) Immunohistochemical comparison of 5-HT in the duodenum of TPH(−/−)-mice (left) and wildtype mice (right). An anti-5-HT-staining is absent in the duodenum of the TPH(−/−)-mice, whereas enterochromaffin cells of TPH(+/+)-mice showed a strong staining.

(G-I) Immunohistochemical comparison of 5-HT (E) and TPH (F) in the nuclei raphe of TPH(−/−)-mice (left) and wildtype mice (right).

(G) Schematic presentation of the cut surface of the vibratome cut. Short additional picture: the vertical line shows the position in a side view of the brain. Large additional picture: Zoom of the stained areas, presented in the same way as in (H) and (I).

4n: Nervus trochlearis or its root; DRD: dorsal nucleus raphe, dorsal part; DRV: dorsal nucleus raphe, ventral part; DRVL: dorsal nucleus raphe, ventrolateral part; mlf: medial longitudinal fascicle; MnR: medial nucleus raphe; PMnR: paramedial nucleus raphe; scp: Pedunculus cerebellaris superior (branchium conjunctivum).

(H) anti-5-HT-staining of TPH(−/−)-mice and TPH(+/+)-mice.

(I) anti-TPH-staining of TPH(−/−)-mice and TPH(+/+)-mice.

FIG. 2:

Sequence and expression of mouse snTPH (Mus musculus).

(A) The sequence comparison between the deduced amino acid sequences of snTPH and pTPH shows the high homology between the two enzymes, with the highest homology being in the catalytic region (aa 149 to 450).

(B) Reversed phase-HPLC-FD chromatograms from the TPH activity assays. (above) nontransfected COS7-cells; (middle) COS7-cells, transiently transfected with a eukaryotic expression vector containing the mouse snTPH-cDNA; (below) P815 mouse mastocytoma cells with a high endogenous TPH-activity. 5-hydroxylated tryptophane (5-HT) was eluted with a residence time of 5 minutes.

(C)RNase protection assay with specific probes for the two isoforms indicated the presence of pTPH mRNA in the duodenum, the thymus gland and the spleen of wildtype animals, but not of TPH(−/−)-mice. snTPH mRNA is exclusively present in the brain, independent of the genotype.

(D) RNase protection assays specific for snTPH (lanes 1-3) and pTPH (lanes 4-6): lane 1: 30 μg brain-RNA, lane 2: 30 μg duodenum-RNA, lane 3: 30 μg P815 RNA, lane 4: 80 μg brain-RNA, lane 5: 80 μg duodenum-RNA, lane 6: 10 μg p815 RNA.

P815-cells express very large amounts of pTPH-mRNA, but no detectable snTPH-mRNA.

(E) The quantitative analysis of snTPH-mRNA in the brain-RNA of wildtype-animals and TPH(−/−)-mice, performed by using the RNase-protection assay, did not show an influence on pTPH-secrease. β-actin mRNA was used as an internal standard.

(F) Comparison of pTPH-mRNA (left) and snTPH-mRNA (right) in the pons- and mid-brain-RNA of wildtype animals. Lane 1-3: 50 μg RNA for pTPH, lane 4-6: 30 μg RNA for snTPH. β-actin mRNA was used as an internal standard.

TABLE 2
sequences of snTPHs according to the present invention

```
SEQ ID No. 1: cDNA-sequence of the neuronal tryptophane hydroxylase (nTPH)
of the human (Homo sapiens)
      1 cattgctctt cagcaccagg gttctggaca gcgccccaag caggcagctg atcgcacgcc
    61 ccttcctctc aatctccgcc agcgctgcta ctgcccctct agtaccccct gctgcagaga
  121 aagaatatta caccgggatc catgcagcca gcaatgatga tgttttccag taaatactgg
  181 gcacggagag ggttttccct ggattcagca gtgcccgaag agcatcagct acttggcagc
  241 tcaacactaa ataaacctaa ctctggcaaa aatgacgaca aaggcaacaa gggaagcagc
  301 aaacgtgaag ctgctaccga aagtggcaag acagcagttg ttttctcctt gaagaatgaa
  361 gttggtggat tggtaaaagc actgaggctc tttcaggaaa aacgtgtcaa catggttcat
  421 attgaatcca ggaaatctcg gcgaagaagt tctgaggttg aaatctttgt ggactgtgag
  481 tgtgggaaaa cagaattcaa tgagctcatt cagttgctga aatttcaaac cactattgtg
  541 acgctgaatc ctccagagaa catttggaca gaggaagaag agctagagga tgtgccctgg
  601 ttccctcgga agatctctga gttagacaaa tgctctcaca gagttctcat gtatggttct
  661 gagcttgatg ctgaccaccc aggatttaag gacaatgtct atcgacagag aagaaagtat
  721 tttgtggatg tggccatggg ttataaatat ggtcagccca ttcccagggt ggagtatact
  781 gaagaagaaa ctaaaacttg gggtgttgta ttccgggagc tctccaaact ctatcccact
  841 catgcttgcc gagagtattt gaaaaacttc cctctgctga ctaaatactg tggctacaga
  901 gaggacaatg tgcctcaact cgaagatgtc tccatgtttc tgaaagaaag gtctggcttc
  961 acggtgaggc cggtggctgg atacctgagc ccacgagact ttctggcagg actggcctac
1021 agagtgttcc actgtaccca gtacatccgg catggctcag atcccctcta caccccagaa
1081 ccagacacat gccatgaact cttgggacat gttccactac ttgcggatcc taagtttgct
1141 cagttttcac aagaaatagg tctggcgtct ctgggagcat cagatgaaga tgttcagaaa
1201 ctagccacgt gctatttctt cacaatcgag tttggccttt gcaagcaaga agggcaactg
1261 cgggcatatg gagcaggact cctttcctcc attggagaat taaagcacgc cctttctgac
1321 aaggcatgtg tgaaagcctt tgacccaaag acaacttgct tacaggaatg ccttatcacc
1381 accttccagg aagcctactt tgtttcagaa agttttgaag aagccaaaga aaagatgagg
1441 gactttgcaa agtcaattac ccgtcccttc tcagtatact tcaatcccta cacacagagt
1501 attgaaattc tgaaagacac cagaagtatt gaaaatgtgg tgcaggacct tcgcagcgac
1561 ttgaatacag tgtgtgatgc tttaaacaaa atgaaccaat atctggggat ttgatgcctg
1621 gaactatgtt gttgccagca tgatcttttt ggggcttagc agcagttcag tcaatgtcat
1681 ataacgcaaa taaccttctg tgtcatggct tggctaataa gcatgcaatt ccatatatct
1741 ataccatctt gtaactcact gtgttagtat ataaagcacc ataagaaatc caatggcaga
1801 taacctgaaa taacgtatta tgtttaaaca tcttaaaaag atttgacatt cctgcttagt
1861 gtccttaacc aaactgcatc tagttaaaat ttgtaacaaa tagccctctt atgagtctca
1921 tttatgccct tttctttttc agatctaagc ctttcctctg tgttcattag ataaaatgaa
1981 aaaaagcagt gaagctgttt ccattttcaa tagtatcagt gttttcacgc attatttgag
2041 ataaacccag aattgtagga aacttcccat cacaataaca aaggttcaat attctatttc
2101 aaaaattgtt gaggtaacac agcagttgga atgattttta ggttgagtat ttacacaatg
2161 caagaaaaca cctttttaca aatggaatta tgtaggttgc gttgaccttg tagaacctga
2221 gttatgacaa gcttcctgaa gtattttgga agatagtact tccggaaagg acattaggaa
2281 agactaaaca gtggacaatc aatcttggga ctatgaattt tatgctggaa taaagtaaat
2341 tatcatgttc
SEQ ID No. 2: Amino acid sequence of the neuronal tryptophane hydroxylase
(nTPH) of the human (Homo sapiens)
MQPAMMMFSSKYWARRGFSLDSAVPEEHQLLGSSTLNKPNSGKNDDKGNKGSSKREAATESGKTAWFSLKNEVGG
LVKALRLFQEKRVNMVHIESRKSRRRSSEVEIFVDCECGKTEFNELIQLLKFQTTIVTLNPPENIWTEEEELEDV
PWFPRKISELDKCSHRVLMYGSELDADHPGFKDNVYRQRRKYFVDVAMGYKYGQPIPRVEYTEEETKTWGWFREL
SKLYPTHACREYLKNFPLLTKYCGYREDNVPQLEDVSMFLKERSGFTVRPVAGYLSPRDFLAGLAYRVFHCTQYI
RHGSDPLYTPEPDTCHELLGHVPLLADPKFAQFSQEIGLASLGASDEDVQKLATCYFFTIEFGLCKQEGQLRAYG
AGLLSSIGELKHALSDKACVKAFDPKTTCLQECLITTFQEAYFVSESFEEAKEKMRDFAKSITRPFSVYFNPYTQ
SIEILKDTRSIENWQDLRSDLNTVCDALNKMNQYLGI
SEQ ID No. 3: cDNA-sequence of the neuronal tryptophane hydroxylase (nTPH)
of the mouse (Mus musculus)
      1 cactgctctt cagcaccagg gttctggaca gcgccccgag caggcagctg ccactgcagt
    61 tcctccttca tctctgccaa ggccgcccct ctggtccccc ctgctgctga gaaagaaaat
  121 tacatcggga gccatgcagc ccgcaatgat gatgttttcc agtaaatact gggccaggag
  181 agggttgtcc ttggattctg ctgtgccaga agatcatcag ctacttggca gcttaacaca
  241 aaataaggct atcaaaagcg aggacaagaa aagcggcaaa gagcccggca aaggcgacac
  301 cacagagagc agcaagacag cagttgtgtt ctccttgaag aatgaagttg gtgggctggt
  361 gaaagcactt agactattcc aggaaaaaca tgtcaacatg cttcatatcg aatccaggcg
  421 gtcccggcga agaagttctg aagtcgaaat cttcgtggac tgcgaatgtg gcaaaacgga
  481 attcaatgag ctcatccagt tgctgaaatt tcagaccacc attgtgaccc tgaatccgcc
  541 tgagagcatt tggacggagg aagaagatct cgaggatgtg ccgtggttcc ctcggaagat
  601 ctctgagtta gacagatgct ctcaccgagt cctcatgtac ggcaccgagc ttgatgccga
  661 ccatccagga tttaaggaca atgtctatcg acagaggagg aagtattttg tggatgtggc
  721 catgggctat aaatatggtc agcccattcc cagggtcgag tacacagaag aagagactaa
  781 aacttggggt gttgtgttcc gggagctctc caaactctac ccgactcatg cttgccggga
  841 gtacctgaaa aacctccccc tgctgaccaa gtactgtggc tacagggaag acaacgtgcc
  901 gcaactggaa gacgtctcca tgtttctgaa agagcgatct ggcttcacag tgagaccagt
  961 ggctggctac ctgagcccaa gagacttcct ggcgggcctg gcctacagag tattccactg
1021 cacccagtac gtgcggcatg gctccgaccc cctctacacc ccggaaccag atacatgcca
1081 tgaactcttg ggacacgtgc cactgcttgc ggatcccaag tttgctcagt tttcccaaga
1141 gataggctta gcgtctctgg gagcctcaga tgaggacgtt cagaaactag ccacgtgcta
1201 tttcttcaca atcgagttcg gcctttgcaa gcaagagggt caactgcggg cgtatggagc
1261 agggttactt tcgtccatcg gagaattgaa gcatgctctt tccgacaagg cgtgtgtgaa
1321 atcctttgac ccaaagacga cctgcttgca ggaatgccta atcaccacct ttcaggacgc
1381 ttactttgtt tcggacagtt ttgaagaagc caaagaaaag atgagggact ttgcaaagtc
1441 aattacccgt cccttctcgg tatacttcaa ccgctacacg cagagcattg aaattctgaa
1501 agacaccaga agtattgaga atgtggtgca ggacctgcgc agtgatttga acacagtgtg
1561 tgatgccttg aataaaatga accaatatct ggggatttga tgcctagaac cagagttatt
1621 gtcagcatga gctcttgggg ggtgtagcaa caatgcagtc aatgttatcc aacatcaaca
1681 actttctgtg tcatggttgg ctagtaagca tgcaattctg tatgtccata cctctgtgta
1741 acttaataac acaaaaatgc tctaaagaac ccatgcagat aaccactcac catttgaaag
1801 attgtgatcc tatttggaca tctcaagtag agttgacatt tctgattagc gaacaaactg
1861 ttaacttaag caaactgtga ctttgaaatc tgtagcaaac attcctcgca caattccagt
1921 cggtgagttg tggaactttt cttccttgga cctgagactt tcctctgtgt tcattagata
1981 aaatgaaaat agttgggagg tggtttctat tttcaatagt atccgtgtta tttgagataa
2041 actagagttg ctccacgctt tgcatcacag caacaaagga tttaatattc tacttcagaa
2101 gctgttcaga aacacagcag ttgggatgga tgtagactga gtgttcagac aatgcaagca
2161 aagaaaagtt ttgataaaca ggatatatag gttgtactga cctcgttgaa accaatttgt
2221 ggcaagcttc ctgaagagct tctggaagga aacacttgaa caaagaatat tcgggaagct
2281 taaacagaag ggatgaaaat cttggaactg tgaatgtatt gttaggatag agtgaattat
2341 cactgcaggc ttttgactcc ttttgcttag actgagaacc tcaaatccca cagggatgta
2401 aataccatct ctgattccaa agagttggag acggagtcgt agagaaacaa agggatttgc
2461 ttcagttagg tctgatgaga tgtgccatgg tcataagcca ctgccctttt atgttggaca
2521 tctgacaagt ctactgtagt gtacatgcat gtttatgtat tgacacagaa agaaaattat
2581 tgcttataaa atgaatgctt ctcaataaac agaatcttgc ccccaaaaaa aaaaaaaa
SEQ ID No. 4: Amino acid sequence of the neuronal tryptophane hydroxylase
(nTPH) of the mouse (Mus musculus)
MQPAMMMFSSKYWARRGLSLDSAVPEDHQLLGSLTQNKAIKSEDKKSGKEPGKGDTTESSKTAWFSLKNEVGGLV
KALRLFQEKHVNMLHIESRRSRRRSSEVEIFVDCECGKTEFNELIQLLKFQTTIVTLNPPESIWTEEEDLEDVPW
FPRKISELDRCSHRVLMYGTELDADHPGFKDNVYRQRRKYFVDVANGYKYGQPIPRVEYTEEETKTWGWFRELSK
LYPTHACREYLKNLPLLTKYCGYREDNVPQLEDVSMFLKERSGFTVRPVAGYLSPRDFLAGLAYRVFHCTQYVRH
GSDPLYTPEPDTCHELLGHVPLLADPKFAQFSQEIGLASLGASDEDVQKLATCYFFTIEFGLCKQEGQLRAYGAG
LLSSIGELKHALSDKACVKSFDPKTTCLQECLITTFQDAYFVSDSFEEAKEKMRDFAKSITRPFSVYFNRYTQSI
EILKDTRSIENWQDLRSDLNTVCDALNKMNQYLGI
SEQ ID No. 5: cDNA-sequence of the neuronal tryptophane hydroxylase (nTPH)
of the rat (Rattus norvegicus)
      1 cagggttctg gacagcgcct cgagcagcca gctgccgctc accttcctcc tacatctctg
    61 ccaaggctgc ccctctgatc ccccctgctg ctgagaaaga aaattacatc gggatccatg
  121 cagcccgcaa tgatgatgtt ttccagtaaa tactgggcca ggagagggtt gtccttggat
  181 tcagcggtgc cagaagagca tcagatactt ggcggcttaa cacaaaataa ggctaccgct
  241 agcaaaagcg aggacaagag aagcggcaaa gacacttcgg agagcagcaa gactgcggtt
  301 gtgttctccc tgaagaatga agttggcggg ctggtgagag cactgagact cttccaggaa
  361 aaacacgtca acatgctcca tattgaatcc aggaggtccc ggcgaagaag ttctgaagtc
  421 gaaatcttcg tggactgtga atgtggcaaa acagaattca acgagctcat tcagttgctc
  481 aagtttcaga ccaccattgt gacgctgaat ccacctgaca acatttggac ggaggaagaa
  541 gaactagagg atgtgccgtg gttccctcgg aagatctctg agttagacag atgctctcac
  601 agagtcctca tgtacggcac cgagcttgac gccgaccacc caggattcaa ggacaacgtc
  661 tatcgacaga ggaggaagta ttttgtggat gtggccatgg gttataaata tggccagccc
  721 attcccaggg tggaatacac agaagaagag actaaaactt ggggtgttgt gtttcgggag
  781 ctctccaaac tctaccccac tcatgcttgc cgagagtacc tgaaaaactt ccccctgctg
  841 accaagtact gcggctacag ggaagacaac gtcccgcagc tggaagacgt ctccatgttt
  901 ctgaaagagc gatctggctt cacagtgaga ccagtggctg gctacctgag cccaagagac
  961 ttcctggctg ggctggccta cagagtattc cactgcactc agtacgtgcg gcatggctcc
1021 gaccccctct acaccccgga accagacaca tgccatgagc tcctgggaca tgtgccactg
1081 ctggcggatc ccaagttcgc tcagttttct caagaaatag gcttagcctc tctgggagcc
1141 tcagatgaag acgttcagaa actggccacg tgctatttct tcacaatcga gttcggcctt
1201 tgcaagcaag aaggtcaact gcgggcgtac ggagcagggt tactttcctc catcggagaa
1261 ttgaagcatg ctctttctga caaggcgtgt gtaaaagcct ttgacccgaa gacaacctgc
1321 ttgcaggaat gcctaatcac caccttccaa gatgcttact ttgtttctga aagttttgaa
1381 gaagccaaag agaagatgag ggattttgca aagtcaatta cccgtccttt ctcagtatat
1441 ttcaacccct acacacagag cattgaaatt ctgaaagaca ccagaagtat tgagaatgtg
1501 gtgcaggacc tgcgcagtga tttgaacacc gtgtgcgacg ccttgaataa aatgaaccaa
1561 tatttgggga tttgagccta ttgtcagcac gagctcttgg gggcttagca acaatgcagt
1621 caatgttatc caacatcaac aactttctgt gtcatggctg gctagtaagc atgcaattcc
1681 atgtgtctat acctctatgt aacttaacat acaaaaatga tctaagaaac ccaggcagat
1741 gaccattcag cattttaaag attgtgatct atttgaacat ctcaagtaga tttgacattt
1801 ctgattagtg agcaaactgt aacttaagca aactgtgtct ttaaaatttg tagccaacat
1861 tcctcacaca attccagctg ctgagtcctt gaccttttct tccttggacc tgagtctttc
1921 ctctgtgttc attagataaa atgaaaacag ttgggaggtg gtttctactt tcaatagtat
1981 tggtgttctc tgagataaac tagagttgct ccaagcttcg catcacagta acaaaagatt
2041 taatatttta cttcagaagc tgttcagaaa cacagcgatt ggaatgaatc tggactgagt
2101 gtttagacaa tgcaagaaaa gaaaaatttt gataaacagg atatatagat tgcactgacc
2161 ttgttgaaac caatttgtgg tacgcttcct gaagtgcttt tggaaggaaa cactttgaca
2221 aagaatattt ggaaaggtta aacagaaggg aagaaaatct tggaactgtg aatgtgtcat
2281 tagaataaag tgaattatca gtgcaggtgt gactcctttc tcttacactg agaaccccaa
2341 atcctgcagg gatgtgagta ccatctctga ttccgaagat ttggaaaccg agtcacagag
2401 aaacaaaggg atttgcttca gttaggtctg ttggctgggg gtgcagtcat aatccccccc
2461 cccctttatg ttggacttct ggcaagtcta ctgtagtgta catgcgggtt tatgtatgga
2521 caaaaaaaga aaactaatgc gtataaaact aatgcttctc aataaacaga aacttgcccc
2581 c
SEQ ID No. 6: Amino acid sequence of the neuronal tryptophane hydroxylase
(nTPH) of the rat (Rattus norvegicus)
MQPAMMMFSSKYWARRGLSLDSAVPEEHQILGGLTQNKATASKSEDKRSGKDTSESSKTAWFSLKNEVGGLVRAL
RLFQEKHVNMLHIESRRSRRRSSEVEIFVDCECGKTEFNELIQLLKFQTTIVTLNPPDNIWTEEEELEDVPWFPR
KISELDRCSHRVLMYGTELDADHPGFKDNVYRQRRKYFVDVAMGYKYGQPIPRVEYTEEETKTWGWFRELSKLYP
THACREYLKNFPLLTKYCGYREDNVPQLEDVSMFLKERSGFTVRPVAGYLSPRDFLAGLAYRVFHCTQYVRHGSD
PLYTPEPDTCHELLGHVPLLADPKFAQFSQEIGLASLGASDEDVQKLATCYFFTIEFGLCKQEGQLRAYGAGLLS
SIGELKHALSDKACVKAFDPKTTCLQECLITTFQDAYFVSESFEEAKEKMRDFAKSITRPFSVYFNPYTQSIEIL
KDTRSIENWQDLRSDLNTVCDALNKMNQYLGI
```

1. An isolated nucleic acid sequence, which encodes a polypeptide with neuronal tryptophane hydroxylase activity, selected from the group of:
a) a nucleic acid sequence with the sequence depicted in SEQ ID No:1, SEQ ID No: 3 or SEQ ID No: 5,
b) a nucleic acid sequence that can be deduced from a nucleic acid sequence depicted in SEQ ID No: 1, SEQ ID No: 3 or SEQ ID No: 5 as a consequence of the degenerated genetic code,
c) derivatives of the nucleic acid sequences depicted in SEQ ID No: 1, SEQ ID No: 3 or SEQ ID No: 5, which encode polypeptides according to SEQ ID No: 2, SEQ ID No: 4 or SEQ ID No: 6, which display at least 80% homology at the amino acid level, wherein the biological activity of the polypeptides is not significantly reduced, and
d) human genomic nucleic acid sequences, which contain the gene for sn-TPH and exhibit polymorphisms.

2. A polypeptide encoded by a nucleic acid sequence according to claim 1.

3. The polypeptide according to claim 2, having the sequence depicted in SEQ ID No: 2, SEQ ID No: 4 or SEQ ID No: 6.

4. A recombinant nucleic acid molecule comprising a nucleic acid sequence according to claim 1 or parts of this nucleic acid sequence, wherein the nucleic acid sequence is connected in an anti-sense or sense-direction with one or several regulatory sequences.

5. A vector comprising a nucleic acid sequence according to claim 1.

6. A recombinant prokaryotic or eukaryotic host organism containing at least one nucleic acid sequence according to claim 1.

7. The recombinant prokaryotic or eukaryotic host organism according to claim 6, wherein the organism is a microorganism or an animal.

8. Use of a polypeptide according to claim 2 or of peptide fragments thereof as an antigen for the production of specific polyclonal or monoclonal antibodies or antibody mixtures.

9. A polyclonal or monoclonal antibody or antibody mixtures, which recognises specific polypeptides according to claim 2.

10. A method for isolating a compound that binds to a polypeptide of claim 2, or of producing a pharmaceutical composition, comprising:
(a) contacting a mammalian cell that expresses the polypeptide of claim 2 having sn-TPH activity with a compound;
(b) detecting the presence of a compound that binds to the sn-TPH polypeptide, and
(c) determining whether the compound binds said sn-TPH polypeptide.

11. The method, according to claim 10, for the production of a pharmaceutical composition comprising the steps of the process according to claim 10 and the subsequent step of formulating the compound identified in step (c) and/or its pharmaceutically acceptable salts in a pharmaceutically acceptable form.

12. A method for the treatment of a neuronal disease in a patient, wherein said method is characterised in that serotonin production in the patient is increased or decreased by affecting the snTPH-activity.

13. The method for the treatment of a neuronal disease according to claim 12, characterised in that the serotonin production is increased by a tissue-specific overexpression of snTPH, by the addition of the precursor substance 5-hydroxy-tryptophane or by the addition of substituted analogues of 5-hydroxy-tryptophane.

14. The method for the treatment of a neuronal disease according to claim 12, characterised in that the serotonin production is decreased by ribozymes, by antisense-oligonucleotides, by antisense-RNA-expression or by means of a specific TPH-inhibitor.

15. A method for determining the pharmacogenetic properties of a pharmaceutically active compound and/or improving treatment of a disease, comprising a) administering the compound to a mammal, b) determining the level of expression of snTPH in a biological sample obtained from said mammal, and c) comparing said level of expression of snTPH with a level obtained from a control sample.

16. The method, according to claim 15, for the improved treatment of a disease, comprising performing the method of claim 15, and increasing or decreasing the doses of the pharmaceutically active compound to be applied to said patient.

17. The method according to claim 16, wherein the disease is selected from the group consisting of neuronal diseases, sleep disturbances, anxiety, alcoholism, drug abuse, disorders of food uptake and sexual disorders.

18. A method for the treatment of sleep disturbances, anxiety, alcoholism, drug abuse, disorders of food uptake or sexual disorders, characterised in that the serotonin level is affected by modulating the gene expression of snTPH wherein said method comprises administering, to a patient in need of such treatment, a nucleotide sequence of claim 1 or a polypeptide of claim 2.

19. A method for diagnosing a neuronal disease, characterised in that a specific inhibition of the peripheral serotonin biosynthesis is accomplished, followed by subsequently detecting the metabolite concentrations stemming from the CNS and by determining the severity of the disease via a comparative graph.

20. Use of a nucleic acid sequence according to claim 1 or of a polypeptide according to claim 2 wherein said use is selected from the group consisting of: identifying/discovering proteins, which have specific binding affinities for a polypeptide according to claim 2 or for identifying nucleic acids, which encode for proteins having specific binding affinities for a polypeptide according to claim 2; the isolation of a genomic sequence by means of homology screening or as a marker for human hereditary diseasees; and gene therapy.

21. The method, according to claim 20, characterised in that the Two-Hybrid-System is employed.

22. (canceled)

23. (canceled)

24. Use of a DNAsequence according to claim 1 or of a polypeptide according to claim 2 for affecting the serotonin level via specific regulation of the snTPH-activity/amount.

25. A combination therapeutic comprising a polypeptide according to claim and at least one additional protein, in particular for the regulation of the serotonin metabolism.

26. The combination therapeutic according to claim 25, characterised in that the additional protein is a peripheral tryptophane hydroxylase.

27. The combination therapeutic according to claim 26, characterised in that the peripheral and the neuronal serotonin production are simultaneously increased or decreased.

28. Use of the combination therapeutic according to claim 25 for the treatment of bleeding episodes in the psycho-pharmacological treatment of depressions with antidepressants, which affect the serotonin reuptake-transporter, containing antidepressants and von Willebrand-factor.
